(19) United States
(12) Patent Application Publication
(10) Pub. No.: US 2022/0091119 A1
(43) Pub. Date: Mar. 24, 2022

(54) Methods for the Diagnosis and Detection of Viral Analytes

(71) Applicant: GEORGIA TECH RESEARCH CORPORATION, Atlanta, GA (US)

(72) Inventor: Jie XU, Marietta, GA (US)

(21) Appl. No.: 17/448,095

(22) Filed: Sep. 20, 2021

Related U.S. Application Data
(60) Provisional application No. 63/080,574, filed on Sep. 18, 2020.
(60) Provisional application No. 63/161,229, filed on Mar. 15, 2021.

(51) Int. Cl. G01N 33/569; G01N 21/45; G01S 19/01; G01N 21/85

(52) U.S. Cl. CPC G01N 33/56983; G01N 21/45; G01N 2201/06113; G01N 21/85; G01N 2333/165; G01S 19/01

(57) ABSTRACT

Methods of detecting one or more viral analytes with an optical interferometric system is provided. Methods of diagnosing a viral infection in a patient with an optical interferometric system are also provided.

### CROSS-REFERENCE TO RELATED APPLICATIONS

The present application claims priority to U.S. Provisional Application No. 63/080,574 filed Sep. 18, 2020 and U.S. Provisional Application No. 63/161,229 filed Mar. 15, 2021.

### BACKGROUND OF THE DISCLOSURE

The COVID-19 pandemic caused by SARS-CoV-2 was a global health emergency. There was an urgent need for rapid and accurate detection of positive infection cases to stop the viral spread. Like other viral tests, different molecular targets are available. Viral nucleic acids or proteins can be used to detect the presence of virus in patients to diagnose acute infection. Detecting and quantifying antibodies that are generated by hosts against the virus can also provide insights into past infection and immunity gained to the disease.

The reverse transcription quantitative polymerase chain reaction (RT-qPCR) test is considered the gold standard for the diagnosis of SARS-CoV-2 and is based on the reverse transcription of the viral RNA into complementary DNA (cDNA) and amplifying a specific region. Although the method is considered highly effective, the RT-PCR based tests are typically performed in centralized laboratories due to the requirement of dedicated equipment, trained personnel, and stringent contamination control.

Humoral immune responses to the SARS-CoV-2 are typically evaluated by enzymatic immunosorbent assays (ELISAs) and its many variants including chemiluminescent based immunoassay and lateral flow based immunoassay (LFA), etc., where viral proteins are immobilized on testing substrate and the binding between the antibody and viral proteins are monitored. Four major structural proteins including the spike (S), membrane (M), envelope (E), and nucleocapsid (N) proteins are found on the surface of coronavirus. The S-protein/host receptor binding followed by proteolytic processing of the viral glycoprotein to promote virus-cell membrane fusion was involved for viral entry. A well-characterized receptor-binding domain (S-RBD) on S-protein attaches angiotensin-converting enzyme 2 (ACE2) specifically as its cellular receptor. Antibodies to S and N protein are the main targets for COVID-19 serologic tests. Particularly, antibodies to S protein and its receptor binding domain (RBD) are the main target for neutralizing antibodies as they prevent the virus binding to epithelial cells in the airway through its entry receptor ACE2. Traditional ELISA uses fluorescent, chemiluminescent or magnetic labeled molecules to produce the detectable signal. The entire process is time and labor intensive, requires multiple washing, incubations steps.

Current methods for blood detection in serum are performed using ELISA (enzyme-linked immunosorbent assay) methods in 96-well plate formats. The testing substrate is conjugated with viral proteins (or peptides). The serum sample is added to the testing substrate. The viral protein-specific antibody (primary antibody) will be bound to the testing surface. The antigen/antibody binding will be reported through an enzyme-labeled secondary antibody. The traditional approach involves multiple steps of rinsing, incubation which is time-consuming, labor-intensive and limited to a lab setting. Therefore, there is a need for rapid detection of viral analytes and diagnosis of viral infection.

### SUMMARY OF THE DISCLOSURE

A method of detecting one or more viral analytes is provided. The method of detecting one or more viral analytes includes the step of analyzing a test sample composition with an optical interferometric system to detect the presence of at least one viral analyte. The optical interferometric system includes an interferometric chip that, in turn, includes one or more waveguide channels having a sensing layer thereon, the sensing layer comprising a plurality of bio-receptors adapted to bind or otherwise be selectively disturbed by one or more viral analytes within the test sample composition. According to one embodiment, the virus is a coronavirus such as alphacoronavirus, betacoronavirus, gammacoronavirus, or deltacoronavirus. According to one embodiment, the coronavirus is a severe acute respiratory syndrome-related coronavirus (SARS-CoV). According to one embodiment, the coronavirus is SARS-CoV-2. According to one embodiment, the plurality of bio-receptors are adapted to covalently bio-conjugate to the sensing layer and bind to viral-specific antibodies. According to one embodiment, the plurality of bio-receptors are adapted to covalently bio-conjugate to the sensing layer through amine coupling. According to one embodiment, the test sample composition includes buffer and a target sample taken from whole blood, blood serum, saliva, mucus, or exhaled breath.

A method of diagnosing a viral infection in a patient is also provided. The method of diagnosing a viral infection includes the step of analyzing a target sample with the interferometric system to detect the presence of at least one viral analyte. When at least one virus detected, a diagnosis of a viral infection is provided. The optical interferometric system includes an interferometric chip that, in turn, includes one or more waveguide channels having a sensing layer thereon, the sensing layer comprising a plurality of bio-receptors adapted to bind or otherwise be selectively disturbed by one or more viral analytes within the test sample composition. According to one embodiment, the virus is a coronavirus such as alphacoronavirus, betacoronavirus, gammacoronavirus, or deltacoronavirus. According to one embodiment, the coronavirus is a severe acute respiratory syndrome-related coronavirus (SARS-CoV). According to one embodiment, the coronavirus is SARS-CoV-2. According to one embodiment, the plurality of bio-receptors are adapted to covalently bio-conjugate to the sensing layer and bind to viral-specific antibodies. According to one embodiment, the plurality of bio-receptors are adapted to covalently bio-conjugate to the sensing layer through amine coupling. According to one embodiment, the test sample composition includes buffer and a target sample taken from whole blood, blood serum, saliva, mucus, or exhaled breath.

An optical inteferometric chip for detecting viral analytes or diagnosing viral analyte infection is also provided. The chip includes at least one waveguide channel having a sensing layer, the sensing layer including a plurality of bio-receptors adapted to bind or otherwise be selectively disturbed by one or more viral analytes. According to one embodiment, the plurality of bio-receptors are adapted to covalently bio-conjugate to the sensing layer and bind to viral-specific antibodies. According to one embodiment, the plurality of bio-receptors are adapted to covalently bio-conjugate to the sensing layer through amine coupling.

### BRIEF DESCRIPTION OF THE DRAWINGS

FIG. 1 illustrates a perspective view of one embodiment of a handheld interferometric system as referred to herein.

FIG. 2 illustrates a front view of one embodiment of a handheld interferometric system as referred to herein.

FIG. 3A illustrates a cross-sectional view of an interferometric chip that may be integrated into a cartridge system as referred to herein.

FIG. 3B illustrates a bottom view of a flow cell wafer having a serpentine shaped detection microchannel.

FIG. 3C illustrates a top view of a chip illustrating the movement of an light signal through the chip.

FIG. 4 is a graph of receptor binding domain (RBD) response over time for a target sample composition taken from convalescent blood serum of a non-human primate.

### DETAILED DESCRIPTION OF THE DISCLOSURE

One or more aspects and embodiments may be incorporated in a different embodiment although not specifically described. That is, all aspects and embodiments can be combined in any way or combination. When referring to the compounds disclosed herein, the following terms have the following meanings unless indicated otherwise. The following definitions are meant to clarify, but not limit, the terms defined. If a particular term used herein is not specifically defined, such term should not be considered indefinite. Rather, terms are used within their accepted meanings.

### Definitions

As used herein, the term “analyte” refers to a substance that is detected, identified, measured or any combination thereof by an interferometric system described herein. The analyte includes a virus, viral protein, viral spike protein, viral antigen or viral antibody.

As used herein the term “virus” refers to an infective agent that can only multiply within a host organism. The virus includes, but is not limited to, any virus that may infect a mammalian host. Exemplary viruses include, but are not limited to, viruses that cause the common cold, influenza, chicken pox, rabies, Ebola, AIDS/HIV, avian influenza, herpes, and severe acute respiratory syndrome (SARS).

As used herein, the term “viral” refers to the nature of, caused by, or relating a virus or viruses.

As used herein, the terms “sample” and “target sample” all refer to any substance that may be subject to the methods and systems referred to herein. Particularly, these terms refer to any matter (animate or inanimate) where an analyte may be present and capable of being detected, quantified, monitored or a combination thereof. Suitable examples of targets include, but are not limited to, whole blood, blood serum, saliva, mucus, and exhaled breath.

As used herein, the term “bio-receptor” refers to any component of a interferometric chip sensing layer that binds or is otherwise be selectively disturbed by one or more viral analytes.

“Optional” or “optionally” means that the subsequently described event or circumstance may or may not occur, and that the description includes instances where said event or circumstance occurs and instances where it does not.

As used herein, the term “buffer” refers to a carrier that is mixed with the target sample that includes at least one analyte.

As used herein, the term “test sample composition” refers to the combination of at least one buffer and target sample.

As used herein, the term “communication” refers to the movement of air, liquid, mist, fog, buffer, test sample composition, or other suitable source capable of carrying an analyte throughout or within the cartridge system. The term “communication” may also refer to the movement of electronic signals between components both internal and external to the cartridge systems described herein.

As used herein, the term “single-use” refers to the cartridge system being utilized in an interferometric system for a single test or assay before disposal (i.e., not re-used or used for a second time).

As used herein, the term “multiple-use” refers to the cartridge system being utilized for more than one test or assay before disposal.

As used herein, the term “multiplex” refers to the cartridge system being utilized to detect multiple analytes from one target sample composition.

The systems and methods referred to herein provide an ultra-sensitive and universal interferometric sensing platform for detecting viral analytes and diagnosing viral infections. The systems and methods referred to herein may be utilized in conjunction with bodily fluids including, but not limited to, whole blood, blood serum, nasal swabs (mucus), and saliva. Once obtained, such bodily fluids may be analyzed via the systems and methods provided herein in an efficient manner to detect one or more viral analytes or provide a diagnosis of a viral infection.

### Optical Interferometry Principles

The systems and methods provided herein may be utilized in optical interferometric systems. Such interferometric systems may include a detector unit that operates via ultrasensitive, optical waveguide interferometry. The waveguiding and the interferometry techniques are combined to detect, monitor and even measure small changes that occur in an optical beam along a propagation pathway. These changes can result from changes in the length of the beam's path, a change in the wavelength of the light, a change in the refractive index of the media the beam is traveling through, or any combination of these, as shown in Equation 1.

$$φ = 2 Π L n / λ \qquad Equation 1$$

According to Equation 1, φ is the phase change, which is directly proportional to the path length, L, and refractive index, n, and inversely proportional to the wavelength (λ) change. According to the systems and methods referred to herein, the change in refractive index is used. Optical waveguides are utilized as efficient sensors for detection of refractive index change by probing near the surface region of the sample with an evanescent field. Particularly, the systems referred to herein can detect small changes in an interference pattern.

The waveguide and interferometer may act independently or in tandem to focus an interferometric diffraction pattern. The waveguide, interferometer, and sensor act may independently or two parts in tandem, or collectively to focus an interferometric pattern with or without mirrors or other reflective or focal median. The waveguide and interferometer may exhibit a coupling angle such that focus at an optimum angle to allow the system to be compact and suited to be portable and hand-held.

### Interferometric System Overview

The interferometric systems referred to herein may accurately provide detection and quantification of viral analytes in a variety of environments. The interferometric systems utilized may provide both qualitative and quantitative results from one or more viral analytes within a test sample composition. Particularly, the systems as referred to herein may simultaneously provide detection and quantification of one or more viral analytes from a test sample composition. According to one embodiment, both qualitative and quantitative results are provided in real-time or near real time.

The interferometric systems referred to herein may include a cartridge system. The cartridge systems referred to herein integrate with one or more independent or integrated optical waveguide interferometers. The cartridge systems provide efficient sample composition communication through a microfluidic system mounted on or within the cartridge housing. The cartridge is suitable for one or more analytes to be detected in a single sample in a concurrent, simultaneous, sequential or parallel manner. The cartridge systems referred to herein may be utilized to analyze in a multiplex manner. That is, one test sample composition will be tested to determine the presence of multiple analytes at the same time by utilizing a plurality of waveguide channels that interact with the test sample composition.

According to one embodiment, the interferometric systems referred to herein have an analyte detection limit down to about 10 picogram/ml. According to one embodiment, the systems referred to herein have an analyte detection limit down to about 1.0 picogram/ml. According to one embodiment, the systems referred to herein have an analyte detection limit down to about 0.1 picogram/ml. According to one embodiment, the systems referred to herein have an analyte detection limit down to about 0.01 picogram/ml. According to one embodiment, the systems referred to herein have an analyte detection limit down to about 0.01 picogram/L.

According to one embodiment, the interferometric systems as referred to herein have an analyte detection limit down to about 3000 plaque forming units per milliliter (pfu/ml). According to one embodiment, the systems as referred to herein have an analyte detection limit down to about 2000 pfu/ml. According to one embodiment, the systems as referred to herein have an analyte detection limit down to about 1000 pfu/ml. According to one embodiment, the systems as referred to herein have an analyte detection limit down to about 500 plaque forming units per milliliter (pfu/ml). According to one embodiment, the systems as referred to herein have an analyte detection limit down to about 100 plaque forming units per milliliter (pfu/ml). According to one embodiment, the systems as referred to herein have an analyte detection limit down to about 10 plaque forming units per milliliter (pfu/ml). According to one embodiment, the systems as referred to herein have an analyte detection limit down to about 1 plaque forming units per milliliter (pfu/ml). According to one embodiment, the systems as referred to herein have an analyte detection limit to about 1 plaque forming units per liter (pfu/l).

The interferometric systems as referred to herein may be equipped with one or more software packages loaded within. The software may be electronically connected to the various system components as referred to herein. The software may also be electronically integrated with a display for viewing by a user. The display may be any variety of display types such as, for example, a LED-backlit LCD. The system may further include a video display unit, such as a liquid crystal display (“LCD”), an organic light emitting diode (“OLED”), a flat panel display, a solid state display, or a cathode ray tube (“CRT”).

According to one embodiment, the interferometric system as referred to herein may interface with or otherwise communicate with a transmission component. The transmission component may be in electronic signal communication with both the cartridge system and interferometric system components. The transmission component sends or transmits a signal regarding analyte detection data and quantification data. The transmission of such data may include real-time transmission via any of a number of known communication channels, including packet data networks and in any of a number of forms, including instant message, notifications, emails or texts. Such real-time transmission may be sent to a remote destination via a wireless signal. The wireless signal may travel via access to the Internet via a surrounding Wi-Fi network. The wireless signal may also communicate with a remote destination via Bluetooth or other radio frequency transmission. The remote destination may be a smart phone, pad, computer, cloud device, or server. The server may store any data for further analysis and later retrieval. The server may analyze any incoming data using artificial intelligence learning algorithms or specialized pathological, physical, or quantum mechanical expertise programed into the server and transmit a signal.

According to one embodiment, the transmission component may include a wireless data link to a phone line. Alternatively, a wireless data link to a building Local Area Network may be used. The system may also be linked to Telephone Base Unit (TBU) which is designed to physically connect to a phone jack and to provide 900 MHz wireless communications thereby allowing the system to communicate at any time the phone line is available.

According to one embodiment, the interferometric system may include a location means. Such a location means includes one or more geolocation device that records and transmits information regarding location. The location means may be in communication with a server, either from a GPS sensor included in the system or a GPS software function capable of generating the location of the system in cooperation with a cellular or other communication network in communication with the system. According to a particular embodiment, the location means such as a geolocation device (such as GPS) may be utilized from within its own device or from a mobile phone or similarly collocated device or network to determine the physical location of the cartridge system.

According to one embodiment, the interferometric system contains a geo-location capability that is activated when a sample is analyzed to “geo-stamp” the sample results for archival purposes. According to one embodiment, the interferometric system contains a time and date capability that is activated when a sample is analyzed to time stamp the sample results for archival purposes.

The interferometric systems referred to herein may interface with software that can process the signals hitting the detector unit. The cartridge system as referred to herein may include a storage means for storing data. The storage means is located on or within the cartridge housing or within the interferometric system housing. The storage means communicates directly with electronic components of the interferometric system. The storage means is readable by the interferometric system. Data may be stored as a visible code or an index number for later retrieval by a centralized database allowing for updates to the data to be delivered after the manufacture of the cartridge system. The storage means may include memory configured to store data referred to herein.

The data retained in the storage means may relate to a variety items useful in the function of the interferometric system. According to a particular embodiment, the data may provide the overall interferometric system or cartridge system status such as whether the cartridge system was previously used or is entirely new or un-used. According to a particular embodiment, the data may provide a cartridge system or interferometric system identification. Such an identification may include any series of letter, numbers, or a combination thereof. Such identification may be readable through a QR code. The identification may be alternatively memorialized on a sticker located on the cartridge housing or interferometric system housing. According to one embodiment, the cartridge housing contains a bar code or QR code. According to one embodiment, the cartridge system contains a bar code or QR code for calibration or alignment. According to one embodiment, the cartridge system contains a bar code or QR code for identification of the cartridge or test assay to be performed.

According to a particular embodiment, the data retained in the storage means may provide the number of uses remaining for a multiple-use cartridge system. According to a particular embodiment, the data may provide calibration data required by interferometric system to process any raw data into interpretable results. According to a particular embodiment, such data may relate to information about the analyte and any special processing instructions that can be utilized by the cartridge system to customize the procedure for the specific combination of receptive surface(s) and analyte(s). The interferometric system as referred to herein may include electronic memory to store data via a code or an index number for later retrieval by a centralized database allowing for updates to the data to be delivered after the manufacture of the cartridge system.

The interferometric system may include a memory component such that operating instructions for the interferometric system may be stored. All data may be stored or archived for later retrieval or downloading onto a workstation, pad, smartphone or other device. According to one embodiment, any data obtained from the system referred to herein may be submitted wirelessly to a remote server. The interferometric system may include logic stored in local memory to interpret the raw data and findings directly, or the system may communicate over a network with a remotely located server to transfer the raw data or findings and request interpretation by logic located at the server. The interferometric system may be configured to translate information into electrical signals or data in a predetermined format and to transmit the electrical signals or data over a wireless (e.g., Bluetooth) or wired connection within the system or to a separate mobile device. The interferometric system may perform some or all of any data adjustment necessary, for example adjustments to the sensed information based on analyte type or age, or may simply pass the data on for transmission to a separate device for display or further processing.

The interferometric systems referred to herein may include a processor, such as a central processing unit (“CPU”), a graphics processing unit (“GPU”), or both. Moreover, the system can include a main memory and a static memory that can communicate with each other via a bus. Additionally, the system may include one or more input devices, such as a keyboard, touchpad, tactile button pad, scanner, digital camera or audio input device, and a cursor control device such as a mouse. The system can include a signal generation device, such as a speaker or remote control, and a network interface device.

According to one embodiment, the interferometric system may include color indication means to provide a visible color change to identify a particular analyte. According to one embodiment, the system may include a reference component that provides secondary confirmation that the system is working properly. Such secondary confirmation may include a visual confirmation or analyte reference that is detected and measured by the detector.

The interferometric system as referred to herein may also include a transmitting component. The transmitting component may be in electronic signal communication with the detector component. The transmitting component sends or transmits a signal regarding analyte detection and quantification data. The transmission of such data may include real-time transmission via any of a number of known communication channels, including packet data networks and in any of a number of forms, including text messages, email, and so forth. Such real-time transmission may be sent to a remote destination via a wireless signal. The wireless signal may travel via access to the Internet via a surrounding Wi-Fi network. The wireless signal may also communicate with a remote destination via Bluetooth or other radio frequency transmission. The remote destination may be a smart phone, pad, computer, cloud device, or server. The server may store any data for further analysis and later retrieval. The server may analyze any incoming data using artificial intelligence learning algorithms or specialized pathological, physical, or quantum mechanical expertise programed into the server and transmit a signal.

According to one embodiment, the system may also include geolocation information in its communications with the server, either from a GPS sensor included in the system or a GPS software function capable of generating the location of the system in cooperation with a cellular or other communication network in communication with the system. According to a particular embodiment, the system may include a geolocation device (such as GPS or RFID) either from within its own device or from a mobile phone or similarly collocated device or network to determine the physical location of the system.

According to one embodiment, the interferometric system includes an external camera. The external camera may be at least partially located within the interferometric system housing but include a lens exposed to the exterior of the housing such that the external camera may take photos and video of a target sample prior to collection (e.g., soil, plant, etc.). The external camera may capture images that aid in the identification of an analyte and confirmation of the resulting data. The external camera may also capture images that aid in selecting a proper remedial measure.

### Handheld Interferometric System—Exemplary Embodiment

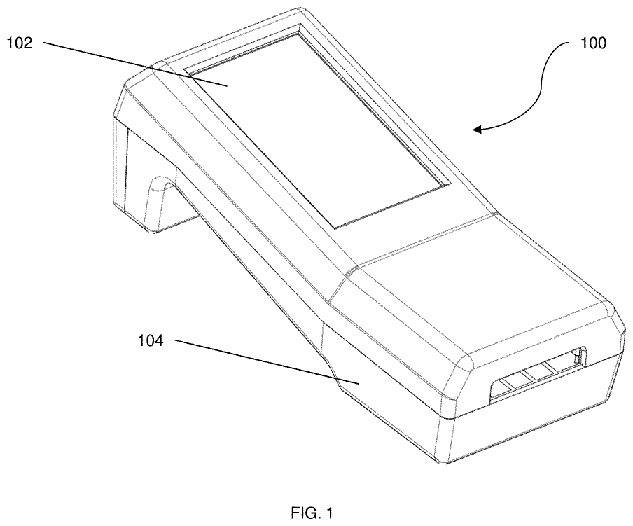

FIG. 1 illustrates a perspective view of one embodiment of an interferometric system 100 that may be utilized to carry out the methods referred to herein. The handheld interferometric system 100 may include a display unit 102. The handheld interferometric system 100 may include a housing 104 adapted to fit within a user's hand.

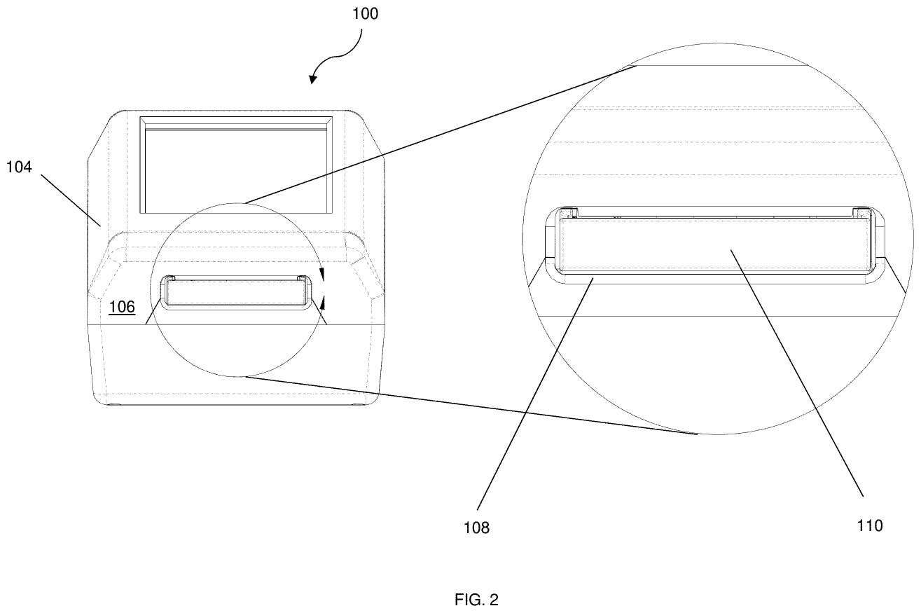

FIG. 2 illustrates a front view of one embodiment of an interferometric system 100 suitable for carrying out the methods provided herein. The housing 104 includes an external front surface 106 defining an opening 108 adapted to receive the cartridge system referred to herein. The opening 108 aids in the alignment and proper position of the cartridge system as referred to herein within the handheld interferometric system 100. The opening 108 may optionally include a flap 110 that shields or covers the opening 108 when the cartridge is not inserted. The flap 110 may be hinged on any side so as to aid in the movement of the flap 110 from a first, closed position to a second, open position upon insertion of the cartridge system.

### Chip, Flow Cell and Optical Assembly—Exemplary Embodiment

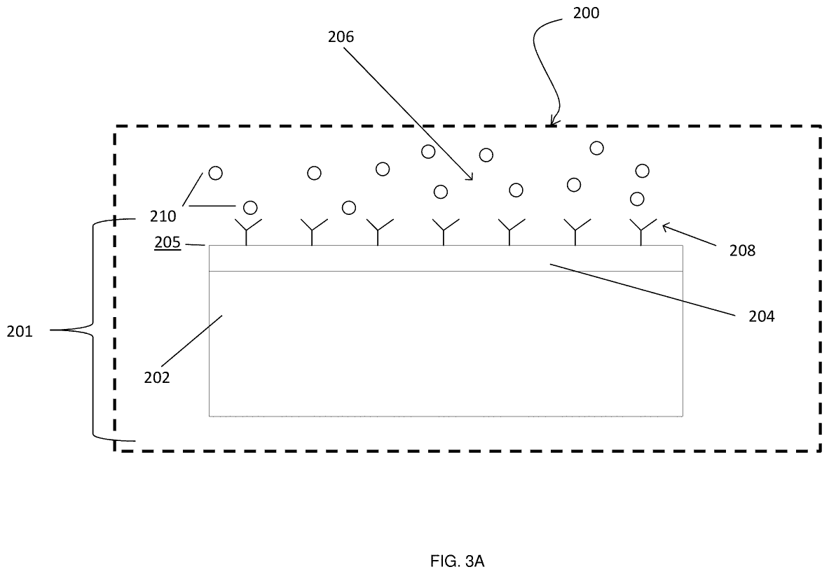

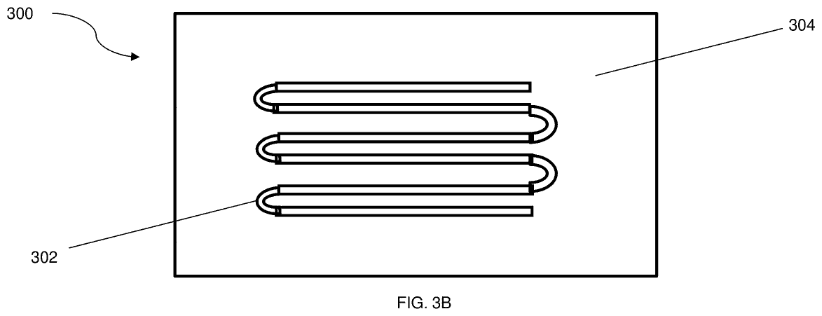

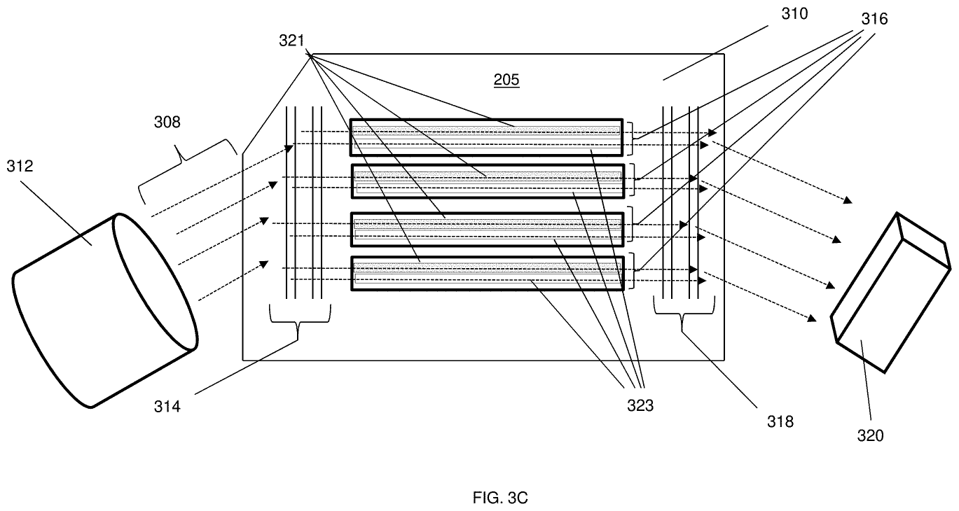

FIG. 3A illustrates a cross-sectional view of an optical detection region 200 of a cartridge system. A chip (or substrate) 202 includes a waveguide channel 204 attached to a surface 205 (such as the illustrated top surface) of the chip 202. The chip 202 may be fabricated from silicon oxynitride.

An evanescent field 206 is located above the waveguide channel 204. A sensing layer 208 is adhered to a top side 205 of the waveguide channel 204. As illustrated, antibodies 210 are shown that may bind or otherwise immobilized to the sensing layer 208, however, the sensing layer 208 may be adapted to bind any variety of analytes. As such, adjusting or otherwise modifying the sensing layer 208 allows for the cartridge system to be utilized for multiple different types of analytes without having to modify the cartridge system or and surrounding interferometric system components. In general use, an light signal (e.g., laser beam) illuminates the waveguide channel 204 creating the evanescent field 206 that encompasses the sensing layer 208. Binding of an analyte impacts the effective index of refraction of the waveguide channel 204.

According to a particular embodiment, the sensing layer 208 may include at least one bio-receptor for binding one or more viral analytes. According to a particular embodiment, the sensing layer 208 may include at least one monoclonal antibody that recognizes and binds a viral analyte such as a viral spike protein. According to another embodiment, the sensing layer 208 may include either antigen or antibody to detect a viral analyte such as an antigen.

A bottom view of an exemplary flow cell 300 is illustrated in FIG. 3B. At least one detection microchannel 302 is located on or within a flow cell 300 manufactured from a wafer. The at least one detection microchannel 302 may be etched, molded or otherwise engraved into one side of the flow cell wafer 304. Thus, the at least one detection microchannel 302 may be shaped as a concave path as a resulted of the etching or molding within the flow cell wafer 304. The flow cell wafer 304 may be manufactured a material such as opaque plastic, or other suitable material. The flow cell wafer 304 may optionally be coated with an anti-reflection composition.

The movement of an light signal 308 (series of arrows) through a chip 310 is illustrated in FIG. 3C. The light signal 308 moves from a light unit 312, such as a laser unit, through a plurality of entry gradients 314 and through one or more waveguide channels 316. Each channel includes a pair of waveguides (321, 323). One of the pair of waveguides 321 is coated with a sensing layer 208 (as indicated by shading in FIG. 3C). The other one of the pair of waveguides 323 is not coated with the sensing layer 208 (serving as a reference). The combination of the light from each in the pair of waveguides (312, 323) create an interference pattern which is illuminated on detector unit 320.

According to a particular embodiment, the two or more waveguides channels 316 are utilized that are able to determine the presence of an analyte that each of the individual waveguides channels 316 alone would not have been able to identify alone. The light signal 308 is then directed by exit gradients 318 to a detector unit 320 such as a camera unit. The detector unit 320 is configured to receive the light signal 308 and detect any analyte present in a target sample composition flowing through the detection microchannel 302 (see FIG. 3B).

The chip 310 includes a combination of substrate 202 (see FIG. 3A), waveguide channel (see FIG. 3A part 204 and FIG. 3C part 316) and sensitive layer 208 (see FIG. 3A). The flow cell 300 is oriented above the top surface 205 of the chip 310 during use such that the detection microchannel 302 may be orientated or otherwise laid out in variety of flow patterns above the waveguide channels 316. The detection microchannel 302 may be laid out, for example, in a simple half loop flow pattern, serial flow pattern, or in a serpentine flow pattern as illustrated in FIG. 3B. The serpentine flow pattern is particularly suited for embodiments where there are multiple waveguide channels 316 that are arranged in a parallel arrangement (see FIG. 3C). By utilizing the serpentine flow pattern, the test composition flows consistently over the waveguide channels 316 without varying flow dynamics.

The light signal passes through each waveguide channel as illustrated in FIG. 3C, may combine thereby forming diffraction patterns on the detector unit. The interaction of the analyte 210 (see FIG. 3A) and the sensing layer 208 changes the index of refraction of light in the waveguide channel per Equation 1. The diffraction pattern is moved which is detected by the detector unit. The detector unit as referred to herein may be in electronic communication with video processing software. Any diffraction pattern movement may be reported in radians of shift. The processing software may record this shift as a positive result. The rate of change in radians that happens as testing is conducted may be proportional to the concentration of the analyte.

### Conjugation Process—Sensing Layer

A bio-conjugation process is provided to covalently immobilize at least one or a plurality of bio-receptors on a sensing layer of one or more waveguide channels. The bio-receptors are adapted to allow for sensitive and selective bindings to one or more viral analytes. To prepare a sensing layer, the top surface of the chip may be initially acid washed. The top surface may then be silanized to produce a monolayer coating with a mercapto functional group. According to one embodiment, surface silanization includes the introduction of silane molecules containing thiol, amine, hydroxyl and/or carboxylic acid functional groups. The concentrations of silanes, contact time and rinsing methods may then be utilized to produce high coverage silanes. Heterogeneous cross-linkers may then be used to interact with the functional groups on the silane molecules and the functional groups in bio-receptors including amine, or thiol groups to conjugate the bio-receptors on the top surface. The concentrations of crosslinkers, solvents (e.g., dimethyl sulfoxide (DMSO), ethanol), contact time and rinsing methods may then be adjusted to produce high coverage crosslinkers on the top surface.

The mercapto coated top surface may then be exposed to a bi-functional crosslinker containing maleimido and hydroxysuccinimide to produce the sensing layer which may include protein conjugation through amine coupling. According to one embodiment, bovine serum albumin, casein and/or superblocks may be used to block the bio-conjugated surface to minimize the non-specific binding sites. Both concentrations of blocking agents and contact time with the bio-conjugated sensing layer may be adjusted to reduce the non-specific binding thereby increasing the targeted bindings. According to one embodiment, protein conjugation with the optimized concentration and binding buffers may be adjusted to produce the most sensitive binding densities of bio-receptors for the sensing layer.

The direct binding between the bio-receptors on the resulting sensing layer and viral analyte may be used to quantify the level of analyte in a test sample composition. The sensitive layer provides a detection sensitivity equal to or greater than conventional ELISA methods. The sensing layer may be fabricated from silicon oxide to achieve an appropriate level of sensitivity.

### Methods of Use—Generally

The systems and methods disclosed herein provide rapid, sensitive, accurate, qualitative and quantitative measurement of one or more viral analytes. According to one embodiment, the methods and systems referred to herein can be used to identify previous exposure to a viral analyte, such as SARS-CoV-2, in recovered patients. According to one embodiment, the methods and systems referred to herein can be used to diagnose a viral infection in previously untested patients. According to one embodiment, the methods and systems referred to herein can be used to diagnose a viral infection in patients that are vaccinated against a particular viral infection. According to one embodiment, the methods and systems referred to herein can be used to diagnose a viral infection in patients that are unvaccinated against a particular viral infection. According to one embodiment, the methods and systems referred to herein can be used to provide a more accurate assessment on the true infection rate in a population. According to one embodiment, the methods and systems referred to herein can be used for viral analyte vaccine efficacy monitoring. According to one embodiment, the methods and systems referred to herein can be used for disease surveillance and outbreak control.

According to a particular embodiment, surface proteins from viral analytes such as SARS-CoV-2, including spike proteins and the receptor binding domain, may be utilized to detect the presence of an antibody specific to a viral analyte. According to one embodiment, spike proteins may be covalently conjugated on the sensing layer to provide the binding sites for coronavirus (e.g., COVID-19) specific antibodies.

According to one embodiment, the methods referred to herein may be carried out in 60 minutes or less without the need for incubation and rinsing. According to one embodiment, the methods referred to herein may be carried out in 30 minutes or less without the need for incubation and rinsing. According to one embodiment, the methods referred to herein may be carried out in 15 minutes or less without the need for incubation and rinsing. According to one embodiment, the methods referred to herein may be carried out in 10 minutes or less. According to one embodiment, the methods referred to herein may be carried out in 5 minutes or less. According to one embodiment, the methods referred to herein may be carried out in 1 minute or less.

### Methods of Detection

A method of detecting one or more viral analytes is provided. The method may include the step of collecting or otherwise obtaining a target sample having one or more viral analytes. In different embodiments, the target sample may be taken from the appropriate target depending on the location and environment. According to a particular embodiment, the target is a mammalian patient such as a human.

According to one embodiment, the method of detecting one or more viral analyte may include the optional step of entering a user identifier (ID) associated with the target sample in the interferometric system. Additionally, an identification number associated with the analyte or interest or a combination thereof may be entered. The cartridge system utilized may be equipped with a label or sticker carrying identifying such information. The label or sticker may include a QR code including such information. The label or sticker may be removed prior to use. Identifying information may include metadata such as time, GPS data, or other data generated by the interferometric system.

According to one embodiment, the method of detecting a viral analyte may optionally include the step of introducing the target sample to the interferometric system. According to one embodiment, target sample is introduced to a cartridge by a separate device such as a syringe or pump. According to one embodiment, target sample is introduced by an injection device. According to one embodiment, the injection device may be permanently attached to a cartridge system. According to one embodiment, the injection device is a pipette. According to one embodiment, the injection device is a syringe. According to one embodiment, the injection device is a lance, pipette or capillary tube.

According to one embodiment, the method of detecting a viral analyte may optionally include the step of mixing the target sample with a buffer solution to form a test sample composition. In a multiple-use cartridge system, such a step may occur prior to the test sample composition being introduced to the cartridge system. In a single-use cartridge system, such a step may occur in the mixing bladder with the assistance of a pump.

The method of detecting a viral analyte includes analyzing the target sample with the interferometric system to detect the presence of at least one viral analyte. Such a step includes initiating waveguide interferometry on the test sample composition. Such a step may include initiating movement of the light signal through the cartridge system as referred to herein and receiving the light signal within the detector unit. Any changes in an interference pattern are representative of viral analyte in the test sample composition. Particularly, such changes in an interference pattern generate data related to one or more viral analyte in the test sample composition. According to one embodiment, the step of initiating waveguide interferometry on the test sample composition includes the step of correlating data from the phase shift with calibration data to obtain data related to viral analyte identity, viral analyte concentration, or a combination thereof.

According to one embodiment, the method of detecting a viral analyte may further include the step of processing any data resulting from changes in the interference pattern. Such changes in interference pattern may be processed and otherwise translated to indicate the presence and amount of viral analyte in a test sample composition. Processing may be assisted by software, processing units, processor, servers, or other component suitable for processing. The step of processing data may further include storing such data in storage means as referred to herein.

According to one embodiment, the method of detecting a viral analyte may optionally include the step of transmitting a data signal . The signal may result in the display of data on the system. The step of transmitting data may include displaying the analyte levels via projecting any real time data on a screen as described herein. The step of transmitting data may include transmitting any obtained data to a mobile phone, smart phone, tablet, computer, laptop, watch or other wireless device. The data may also be sent to a device at a remote destination. The remote destination device may be a locally operated mobile or portable device, such as a smart phone, tablet device, pad, or laptop computer. The destination may also be smart phone, pad, computer, cloud device, or server. In other embodiments, the remote destination may be a stand-alone or networked computer, cloud device, or server accessible via a local portable device.

The method of detecting a viral analyte may optionally include the step of disposing of the test sample composition per legal requirements. Such legal requirements assure that any sample still containing unacceptable levels of pathological contamination are disposed of properly so as not to cause harm to a user or the environment.

### Methods of Diagnosis

A method of diagnosing a viral infection in a patient is provided. The method may include the step of collecting or otherwise obtaining a target sample having one or more viral analytes. In different embodiments, the target sample may be taken from the appropriate target depending on the location and environment. According to a particular embodiment, the target is a mammalian patient such as a human.

According to one embodiment, the method of diagnosing a viral infection further includes the optional step of entering a user identifier (ID) associated with the target sample in the interferometric system. Additionally, an identification number associated with the analyte or interest or a combination thereof may be entered. The cartridge system utilized may be equipped with a label or sticker carrying identifying such information. The label or sticker may include a QR code including such information. The label or sticker may be removed prior to use. Identifying information may include metadata such as time, GPS data, or other data generated by the interferometric system.

According to one embodiment, the method of diagnosing a viral infection optionally includes the step of introducing the target sample to the interferometric system. According to one embodiment, target sample is introduced to the cartridge by a separate device such as a syringe or pump. According to one embodiment, target sample is introduced by an injection device. According to one embodiment, the injection device may be permanently attached to the cartridge system. According to one embodiment, the injection device is a pipette. According to one embodiment, the injection device is a syringe. According to one embodiment, the injection device is a lance, pipette or capillary tube. When utilizing a multiple-use cartridge system, the cartridge system may be fitted to a tube or other transfer mechanism to allow the sample to be continuously taken from a large amount of fluid that is being monitored.

According to one embodiment, the method of diagnosing a viral infection optionally includes the step of mixing the target sample with a buffer solution to form a test sample composition. In a multiple-use cartridge system, such a step may occur prior to the test sample composition being introduced to the cartridge system. In a single-use cartridge system, such a step may occur in the mixing bladder with the assistance of a pump.

The method of diagnosing a viral infection includes analyzing the target sample with the interferometric system to detect the presence of at least one viral analyte. Such a step includes initiating waveguide interferometry on the test sample composition. Such a step may include initiating movement of the light signal through the cartridge system as referred to herein and receiving the light signal within the detector unit. Any changes in an interference pattern are representative of viral analyte in the test sample composition. Particularly, such changes in an interference pattern generate data related to one or more viral analyte in the test sample composition. According to one embodiment, the step of initiating waveguide interferometry on the test sample composition includes the step of correlating data from the phase shift with calibration data to obtain data related to viral analyte identity, viral analyte concentration, or a combination thereof.

According to one embodiment, the method of diagnosing a viral infection may optionally include the step of processing any data resulting from changes in the interference pattern. Such changes in interference pattern may be processed and otherwise translated to indicate the presence and amount of a viral analyte in a test sample composition. Processing may be assisted by software, processing units, processor, servers, or other component suitable for processing. The step of processing data may further include storing such data in storage means as referred to herein.

According to one embodiment, the method of diagnosing a viral infection may optionally include the step of transmitting a data signal. The signal may result in the display of data on the system. The step of transmitting data may include displaying the viral analyte levels via projecting any real time data on a screen as described herein. The step of transmitting data may include transmitting any obtained data to a mobile phone, smart phone, tablet, computer, laptop, watch or other wireless device. The data may also be sent to a device at a remote destination. The remote destination device may be a locally operated mobile or portable device, such as a smart phone, tablet device, pad, or laptop computer. The destination may also be smart phone, pad, computer, cloud device, or server. In other embodiments, the remote destination may be a stand-alone or networked computer, cloud device, or server accessible via a local portable device.

According to one embodiment, the method of diagnosing a viral infection may optionally include displaying the results from the analysis of the test sample composition. When at least one virus is detected, a diagnosis of a viral infection is provided and may be transmitted and displayed as referred to herein. The diagnosis may be submitted to a third party such as an attending physician or government organization.

The method of diagnosing a viral infection may optionally include the step of disposing of the test sample composition per legal requirements. Such legal requirements assure that any sample still containing unacceptable levels of pathological contamination are disposed of properly so as not to cause harm to a user or the environment.

Although the present specification describes components and functions that may be implemented in particular embodiments with reference to particular standards and protocols, the invention is not limited to such standards and protocols. Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be apparent to those skilled in the art, and all modifications that do not depart from the spirit of the disclosure are intended to be included with the scope of the appended claims.

### EXAMPLE 1

### SARS-CoV-2 Specific Antibody Detection

Waveguide interferometry for SARS-CoV-2 specific antibody detection in clinical blood serum samples was performed. Four major structural proteins including the spike (S), membrane (M), envelope (E), and nucleocapsid (N) proteins are found on the surface of coronavirus. Nucleocapsid protein (NP) or different domains of the spike glycoprotein (S1, S2 and receptor binding domain (RBD)) have been used as the bio-receptors for COVID-19 antibody detection. The N protein of coronaviruses is a structural component of the helical nucleocapsid and the spike protein appears to be the primary protein interacting with host cells. Spike protein and RBD were selected as the bio-receptor for assay development because (1) antibodies bind to RBD or spike protein is directly related with antibody neutralization capabilities. The detection method was shown to have high sensitivity (92.5%) and specificity (100%).

The selected bio-receptors (RBD and spike protein—commercially available from BEI Resources) were covalently conjugated on the waveguide surface through amine coupling chemistry as provided herein. Phosphate buffer saline containing 1% of BSA was used as the running buffer. Serum samples were diluted in the running buffer before the test. Pooled Non-Human Primate Convalescent Serum to SARS-CoV-2 (BEI Resources Catalog #NR-52401) was used a positive control for assay development. Running buffer was flowed over the sensing layer for a few minutes to register baseline before the test sample composition was introduced. The sensing data was collected for 5 another 5 to 10 minutes.

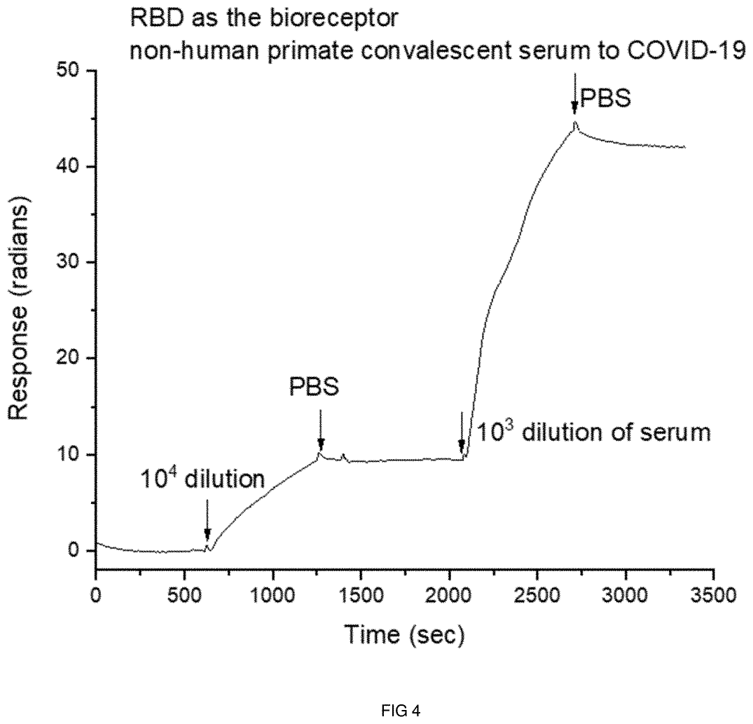

RBD and spike protein coated chips produced a significant binding to the convalescent serum (shown with arrow in FIG. 4). Detection limit was between 100,000 and 10,000 dilution of the blood serum. The method was rapid. Detectable signal was observed right after the sample was introduced into the flow cell. The binding signal was also concentration-dependent so quantitative antibody determination in serum was obtained. In addition, the binding strength could also be estimated based on the association rate and dissociation rate.

No cross bindings were observed for anti-serum samples against IgG depleted serum, H1N1, RSV, H5N1, etc. A total of 13 positive and 10 negative clinical samples were obtained and tested on a waveguide interferometer. The rate of change, by measuring the slope between 100 to 300 sec in the sensing curve (after the sample was introduced into the flow cell), was used to determine the sensor threshold to distinguish from positive from negative samples. The rate of phase change was used due to its independence of initial spikes caused by the bulk refractive index change when the clinical samples were introduced. With a threshold value of 4×10⁻⁴, 12 out of 13 positives and 10 out of 10 negatives were detected. Further, 1×10⁶ copies/ml viral particle in 10 minutes was detected. Thus, the versatility of the system and methods provided herein was demonstrated.

### EXAMPLE 2

### Bio-Conjugation Efficiency Quantification

The bio-conjugation efficiency of a sensing layer was quantified and optimized using fluorescent labeled maleimide or proteins. The impact of different rinsing methods after mercaptopropyltrimethoxysilane (MTS) treatment was investigated first to understand the surface oxidation on the coated MTS. In this example, chips were coated with in 2% MTS solution prepared in toluene for 1 hour. Four different post-MTS rinsing methods were tested as follows:
(a) toluene rinse then followed with air blow-dry before maleimide coupling;
(b) toluene rinse followed with a DMSO rinse before maleimide coupling;
(c) toluene rinse, air blow-dry, followed with a brief TCEP (tris(carboxyethyl)phosphine) soaking for 15 min and then methanol rinsed before maleimide coupling; and
(d) toluene rinse followed with a brief mercaptoethanol soaking and a methanol rinse before maleimide coupling.

After the post-MTS rinsing step, the chips were applied with a drop of maleimide dye and incubated for one hour in the dark. After dye conjugation, the chips were rinsed with DMSO, ethanol and then dried with an airflow before the fluorescence measurement. The fluorescent intensity was compared. The chips exposed to air (air dry) had the lowest fluorescent intensity compared to the other three methods. While not being bound to a particular theory, fluorescent intensity difference could be caused by reduced available thiol binding sites due to the surface oxidation. TCEP treated chips produced the highest fluorescent intensity but also provided inconsistent results. Chips treated with ethanol rinse or mercaptoethanol rinse produced similar fluorescent intensities. To make the conjugation process simpler, post-MTS treatment with toluene followed by DMSO rinse was selected.

The impact of MTS concentration on the fluorescent intensity was investigated next to identify the optimized MTS concentration. MTS solutions with concentrations ranging from 2% to 4% were prepared and tested. Chips incubated with 2% MTS and followed with a toluene rinse and an air blow-dry had a lower fluorescent intensity. The chips incubated with 3% or 4% MTS solutions produced similar fluorescent intensity. A similar study on MTS concentrations in the range of 0.5% to 10% using the entire MTS/N-Succinimidyl 3-Maleimidobenzoate (MBS) and fluorescent protein conjugation process was conducted. Based on analysis of variance (ANOVA), only the chips incubated with 0.5% MTS produced significant difference compared to the rest. Therefore, 3% MTS was shown to be appropriate for MTS conjugation.

The impact of MTS incubation length on the fluorescent intensity was also examined. Chips were incubated with 3% MTS with incubation durations ranging from 0.5 h to 4 h. After MTS incubation, chips were rinsed with toluene and followed with a DMSO rinse before maleimide dye conjugation. Chips with 30 minutes contact time in MTS solution had the lowest fluorescent intensity while the chips incubated with 1 h to 4 h had similar intensities. Therefore, contact time of 1 h was shown appropriate for MTS conjugation.

Both concentration and contact time of MBS were optimized and fluorescent labeled protein was used for conjugation efficiency optimization. Based on the results, the concentration of 2 mM MBS with one hour of contact time was shown appropriate for MBS cross-linking.

The concentrations of fluorescent protein for immobilization was also investigated. The stock protein solution of 2 mg/ml was diluted in phosphate buffered saline (PBS) to make 0.01, 0.1, 0.5 and 1 mg/ml of protein solutions. The diluted protein solution with concentration of 0.1 mg/ml produced the highest fluorescent intensity. In sum, the concentration of bio-receptors in the range of 0.1 mg/ml to 0.5 mg/ml produced highest detection sensitivities.

I/We claim:

1. A method of detecting at least one viral analyte, the method comprising the steps of:
analyzing a test sample composition with an optical interferometric system to detect the presence of at least one viral analyte,
wherein the optical interferometric system comprises an interferometric chip that includes one or more waveguide channels having a sensing layer thereon, the sensing layer comprising a plurality of bio-receptors adapted to bind or otherwise be selectively disturbed by one or more viral analytes within the test sample composition.

2. The method of claim 1, wherein the virus is a coronavirus selected from the group consisting of alphacoronavirus, betacoronavirus, gammacoronavirus, and deltacoronavirus.

3. The method of claim 2, wherein the coronavirus is a severe acute respiratory syndrome-related coronavirus (SARS-CoV).

4. The method of claim 3, wherein the coronavirus is SARS-CoV-2.

5. The method of claim 1, wherein the plurality of bio-receptors are adapted to covalently bio-conjugate to the sensing layer and bind to viral-specific antibodies.

6. The method of claim 5, wherein the plurality of bio-receptors are adapted to covalently bio-conjugate to the sensing layer through amine coupling.

7. The method of claim 1, wherein the test sample composition includes buffer and a target sample taken from whole blood, blood serum, saliva, mucus, or exhaled breath.

8. A method of diagnosing a viral infection in a patient, the method comprising the steps of:
analyzing a target sample with the interferometric system to detect the presence of at least one viral analyte,
wherein upon when at least one virus detected, a diagnosis of a viral infection is provided, and
wherein the optical interferometric system comprises an interferometric chip that includes one or more waveguide channels having a sensing layer thereon, the sensing layer comprising a plurality of bio-receptors adapted to bind or otherwise be selectively disturbed by one or more viral analytes within the test sample composition.

9. The method of claim 8, wherein the virus is a coronavirus selected from the group consisting of alphacoronavirus, betacoronavirus, gammacoronavirus, and deltacoronavirus.

10. The method of claim 9, wherein the coronavirus is a severe acute respiratory syndrome-related coronavirus (SARS-CoV).

11. The method of claim 10, wherein the coronavirus is SARS-CoV-2.

12. The method of claim 8, wherein the plurality of bio-receptors are adapted to covalently bio-conjugate to the sensing layer and bind to viral-specific antibodies.

13. The method of claim 8, wherein the plurality of bio-receptors are adapted to covalently bio-conjugate to the sensing layer through amine coupling.

14. The method of claim 8, wherein the test sample composition includes buffer and a target sample taken from whole blood, blood serum, saliva, mucus, or exhaled breath.
